(19) United States
(12) Patent Application Publication
(10) Pub. No.: US 2009/0171454 A1
(43) Pub. Date: Jul. 2, 2009

(54) COATED STENT AND METHODS OF MANUFACTURE

(75) Inventor: Fred T. Parker, Unionville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/337,863

(22) Filed: Dec. 18, 2008

Related U.S. Application Data
(60) Provisional application No. 61/016,954, filed on Dec. 27, 2007.

(51) Int. Cl. A61F 2/06; A61B 19/00

(52) U.S. Cl. 623/1.46; 623/1.15; 623/1.39; 623/1.12; 128/898

(57) ABSTRACT

The present embodiments provide a coated stent for use in a medical procedure that comprises a coating and a stent disposed over the coating. The stent has a first expanded state in which an inner diameter of the stent is less than or equal to an outer diameter of the coating, thereby causing an inner surface of the stent to engage the outer surface of the coating. The stent may comprise a shape-memory material that is preconfigured to expand to the first expanded state. In one example, the stent may be temporarily held in a second expanded state having an inner diameter larger than the outer diameter of the coating, placed over the coating in the second expanded state, and then allowed to return to the first expanded state to engage the coating.

### PRIORITY CLAIM

This invention claims the benefit of priority of U.S. Provisional Application Ser. No. 61/016,954, entitled “Coated Stent and Methods of Manufacture,” filed Dec. 27, 2007, the disclosure of which is hereby incorporated by reference in its entirety.

### BACKGROUND

The present invention relates generally to apparatus and methods for treating medical conditions, and in particular, to a stent coupled to a coating and being suitable for treating various medical conditions.

Stents may be inserted into an anatomical vessel or duct for various purposes. Stents often are used to maintain or restore patency in a formerly blocked or constricted passageway, for example, following a balloon angioplasty procedure. Some stents may be used in conjunction with a suitable coating to form a coated stent, for example, to treat an aneurysm or to deliver therapeutic agents disposed on the stent or coating in close proximity to a target site.

Stents may be either self-expanding or balloon-expandable, or they can have characteristics of both types of stents. Self-expanding stents may be delivered to a target site in a compressed configuration and subsequently expanded by removing a delivery sheath, removing trigger wires and/or releasing diameter reducing ties. In a stent made of a shape-memory alloy such as nitinol, the shape-memory alloy may be employed to cause the stent to return to a predetermined configuration upon removal of the sheath or other device maintaining the stent in its predeployment configuration.

With balloon-expandable stents, the stent may be delivered and deployed using a catheter having proximal and distal ends and one or more balloons disposed on the catheter. The stent may be coupled to the balloon during insertion until the target site is reached, and then deployed by inflating the balloon to expand the stent to bring the stent into engagement with the target site. Alternatively, the stent may be placed separately in the vessel and a subsequent catheter having an expansion portion may then be inserted into the stent to expand the stent at the target site.

When stents are used in conjunction with a coating, undesirable gaps may be formed between the stent and the coating. To reduce this problem, a coated stent typically comprises a first coating disposed internal to the stent and a second coating disposed external to the stent. Therefore, the stent is sandwiched between the first and second coatings to reduce or eliminate gap formation.

However, where first and second coatings are used, the profile of the stent is increased by at least one additional layer, which may make it difficult to use the stent in smaller vessels or ducts. Moreover, if first and second coatings are employed, it may increase the deployment forced needed to deploy the stent.

In view of the above, it would be desirable to provide a coated stent having little or no gaps formed between the stent and the coating, having a reduced profile, and which facilitates a reduction in deployment force.

### SUMMARY

The present embodiments provide a coated stent for use in a medical procedure that comprises a coating and a stent disposed over the coating. The coating comprises an outer surface having an outer diameter. The stent has a first expanded state having an inner diameter that is less than or equal to the outer diameter of the coating, thereby causing an inner surface of the stent to engage, and at least partially embed into, the outer surface of the coating. The outer surface of the stent may remain substantially or completely free of any coating.

In one example, the coating may comprise a biocompatible polymeric material. For example, the coating may comprise Thoralon®. The stent may comprise a shape-memory material, such as a nickel-titanium alloy. The shape-memory material may be preconfigured to expand to the first expanded state.

The stent may comprise a second expanded state to facilitate placement of the stent over the coating. In the second expanded state, the inner diameter of the stent is greater than the outer diameter of the coating, thereby facilitating co-axial alignment of the stent over the coating. In one example, the stent may achieve an increased diameter in the second expanded state using a balloon catheter.

The stent may be temporarily retained in the second expanded state, then exposed to a reduced temperature environment. For example, the stent may be sprayed with a cooling substance, exposed to liquid nitrogen, immersed in a bath of a cooling substance and/or exposed to a relatively cold ambient temperature. The exposure to the reduced temperature environment may facilitate retention of the stent in the increased diameter of the second expanded state.

In a next step, the stent may be placed over the coating in the second expanded state, then allowed to return to the first expanded state to cause an inner surface of the stent to engage the outer surface of the coating. The stent may return to the first expanded state after being introduced into an increased temperature environment, relative to the reduced temperature environment. For example, the increased temperature environment may cause a shape-memory material to be heated to a transition temperature that returns the stent to the preconfigured first expanded state.

Advantageously, since the inner diameter of the stent in the first expanded state is less than or equal to the outer diameter of the coating, an inner surface of the stent may engage, and at least partially embed into, the outer surface of the coating. If the coating comprises Thoralon®, which may be soft and relatively sticky, then the inner surface of the stent may embed into and securely engage the coating to reduce or eliminate gap formation between the stent and the coating. Moreover, since the stent may be securely disposed over or in the coating with little or no gap formation, application of a second coating over the stent may not be necessary.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a side view of a coated stent.

FIG. 2 is a side view of a balloon catheter being used to expand the stent of FIG. 1.

FIG. 3 is a side view of a coating disposed on a mandrel.

FIG. 4 is a side view of the stent of FIG. 2 being disposed over the coating and the mandrel of FIG. 3.

FIG. 5 is a side view depicting the engagement of the stent with the coating.

### DETAILED DESCRIPTION OF THE PREFERRED EMBODIMENTS

In the present application, the term “proximal” refers to a direction that is generally closest to the heart during a medical procedure, while the term “distal” refers to a direction that is furthest from the heart during a medical procedure.

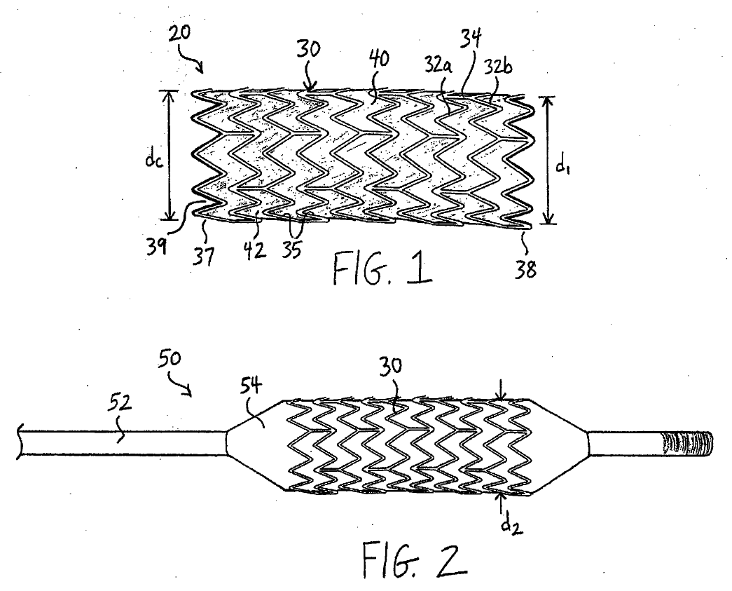

Referring now to FIG. 1, a coated stent 20 is shown after manufacture, preferably according to one or more of the techniques described hereinbelow. The coated stent 20 comprises at least one stent 30 and a coating 40. As used in the present application, the terms “coated” and “coating” generally refer to the provision of one or more layers of material that are separate from the stent itself. The “coating” need not be disposed external to the stent 30, and in the examples described herein, the coating 40 is generally disposed internal to the stent 30, or the stent may be at least partially embedded in the coating 40, as depicted in FIG. 1 and explained further below.

The coated stent 20 may be used in a wide range of procedures, for example, to treat an aneurysm, stenosis or other condition. The stent 30 generally provides radial force needed to expand the coated stent 20 into engagement at a target site, while the coating 40 may provide a barrier having a selected porosity and may be suitable for delivering one or more therapeutic agents, as explained further below. A lumen 39 may be formed internal to the coating 40 and may be suitable for carrying fluid though the coated stent 20.

The stent 30 may be made from numerous metals and alloys. In one example described further below, the stent 20 comprises a shape-memory material such as a nickel-titanium alloy (“nitinol”). Moreover, the structure of the stent 30 may be formed in a variety of ways to provide a suitable intraluminal support structure. For example, one or more stents 30 may be made from a woven wire structure, a laser-cut cannula, individual interconnected rings, or another pattern or design.

In one example, the stent 30 may be configured in the form of one or more “Z-stents” or Gianturco stents, each of which may comprise a series of substantially straight segments interconnected by a series of bent segments. The bent segments may comprise acute bends or apices. The Gianturco stents are arranged in a zigzag configuration in which the straight segments are set at angles relative to each other and are connected by the bent segments.

In the examples shown herein, the structure of stent 30 is similar to the commercially available ZILVER® stent, manufactured by Cook Incorporated of Bloomington, Ind. As shown in FIG. 1, the stent 30 may be formed from a slotted tube generally comprising a series of adjacent segments 32a and 32b and a pattern of connecting segments 34 disposed therebetween. One or more eyelets (not shown) may extend from the proximal end 37 and the distal end 38 of the stent 30, and the eyelets may include a radiopaque material such as gold to provide radiographic visualization of the stent's position when placed in the vessel or duct of a patient. However, as noted above and explained further below, the stent 30 may comprise any suitable configuration and one or more stents may be provided.

The coating 40 may comprise a polymeric sheet having any suitable porosity. The porosity may be substantially porous or substantially non-porous and may be selected depending on the application. In one example, a porous polymeric sheet may comprise the polyurethane Thoralon®. As described in U.S. Patent Application Publication No. 2002/0065552 A1, incorporated herein by reference in its entirety, Thoralon® is a polyetherurethane urea blended with a siloxane-containing surface modifying additive. Specifically, the polymer is a mixture of base polymer BPS-215 and an additive SMA-300. The concentration of additive may be in the range of 0.5% to 5% by weight of the base polymer. The BPS-215 component (Thoratec® Corporation, Pleasanton, Calif.) is a segmented polyether urethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED). The SMA-300 component (Thoratec® Corporation, Pleasanton, Calif.) is a polyurethane comprising polydimethylsiloxane as a soft segment and the reaction product of MDI and 1,4-butanediol as a hard segment. A process for synthesizing SMA-300 is described, for example, in U.S. Pat. Nos. 4,861,830 and 4,675,361, which are incorporated herein by reference. A porous polymeric sheet can be formed from these two components by dissolving the base polymer and additive in a solvent such as dimethylacetamide (DMAC) and solidifying the mixture by solvent casting or by coagulation in a liquid that is a non-solvent for the base polymer and additive.

Thoralon® has been used in certain vascular applications and is characterized by thromboresistance, high tensile strength, low water absorption, low critical surface tension, and good flex life. Thoralon® is believed to be biostable and to be useful in vivo in long term blood contacting applications requiring biostability and leak resistance. Because of its flexibility, Thoralon® may be useful in larger vessels, such as the abdominal aorta, where elasticity and compliance are beneficial.

Further, Thoralon® may also be used as a drug delivery vehicle, for example, to deliver one or more therapeutic agents. The therapeutic agents may be coated onto or contained within a porous outer layer of the coating 40 for sustained release subsequent to an implantation procedure and may be used, for example, to promote intimal cell in-growth.

While Thoralon® is generally described herein, the coating 40 may comprise other materials. In addition to, or in lieu of, a porous polyurethane such as Thoralon®, the coating 40 may comprise any biocompatible polymeric material including non-porous polyurethanes, PTFE, expanded PTFE (ePTFE), polyethylene tetraphthalate (PET), aliphatic polyoxaesters, polylactides, polycaprolactones, and hydrogels. The coating also may comprise a graft material, such as Dacron®, which may optionally be heat treated and/or partially melted.

The stent 30 has a compressed, reduced diameter delivery state in which the coated stent 20 may be advanced to a target location within a vessel, duct or other anatomical site. The stent 30 further has a first expanded state, as shown in FIG. 1, in which it may be configured to apply a radially outward force upon the vessel, duct or other target location, e.g., to maintain patency within a passageway. In the first expanded state, fluid flow is allowed through the lumen 39 of the coated stent 20.

The stent 30 may comprise predetermined inner and outer diameters in the first expanded state. The outer diameter of the stent in the first expanded state may be sized for a particular purpose, e.g., to engage an inner wall of a vessel or duct. As shown in FIG. 1, and explained further below, the inner diameter d₁ of the stent 30 may be sized for snug engagement with an exterior surface 42 of the coating 40. In one example, the predetermined inner diameter d₁ of the stent 30 is less than or equal to an outer diameter d_{c} of the coating 40.

If the stent 30 comprises a shape-memory material such as nitinol, the stent may be manufactured such that it can assume the preconfigured first expanded inner diameter d₁ upon application of a certain cold or hot medium. More specifically, a shape-memory material may undergo a substantially reversible phase transformation that allows it to “remember” and return to a previous shape or configuration. For example, in the case of nitinol, a transformation between an austenitic phase and a martensitic phase may occur by cooling and/or heating (shape memory effect) or by isothermally applying and/or removing stress (superelastic effect). Austenite is characteristically the stronger phase and martensite is the more easily deformable phase.

In an example of the shape-memory effect, a nickel-titanium alloy having an initial configuration in the austenitic phase may be cooled below a transformation temperature (M_{f}) to the martensitic phase and then deformed to a second configuration. Upon heating to another transformation temperature (A_{f}), the material may spontaneously return to its initial, predetermined configuration. Generally, the memory effect is one-way, which means that the spontaneous change from one configuration to another occurs only upon heating. However, it is possible to obtain a two-way shape memory effect, in which a shape memory material spontaneously changes shape upon cooling as well as upon heating.

Referring now to FIGS. 2-5, one or more techniques suitable for manufacturing the coated stent 20 of FIG. 1 are described. In a first step, the bare stent 30 may be formed from a shape-memory material configured to “remember” and return to the preconfigured, first expanded state of FIG. 1, as generally explained above. In a next step, the bare stent 30 may be expanded to a shape that is larger than its predetermined, first expanded state. As explained below, the stent 30 may be expanded to an inner diameter d₂ that is larger than an exterior diameter d_{c} of the coating 40.

In one example, shown in FIG. 2, the stent 30 may be expanded to a second expanded state using a balloon catheter 50. Balloon catheter 50 may comprise a flexible, tubular member 52 having a balloon 54 coupled thereto. The tubular member 52 may be formed from one or more semi-rigid polymers and the balloon 54 may be manufactured from any suitable balloon material used during an interventional procedure, such as PEBAX, nylon, Hytrel, Arnitel, or other polymers. The balloon 54 comprises uninflated and inflated states. The stent 30 may be placed over the balloon 54 and aligned with the balloon when the balloon is in the uninflated state. Subsequently, an inflation fluid may be provided through a lumen of the tubular member 52 and into the inner confines of the balloon 54 to expand the balloon to the inflated state, as shown in FIG. 2. The stent 30 therefore may be expanded to the second expanded state having a predetermined inner diameter d₂, as shown in FIG. 2.

As noted above, the stent 30 may comprise a shape-memory alloy, such as nitinol, that has been treated and configured to return to a predetermined inner diameter d₁. The balloon 54 may mechanically expand the diameter of the stent 30 to a larger inner diameter d₂, which is beyond its heat-set inner diameter d₁, as depicted in FIG. 2. As long as the strain imposed upon the stent 30 is less than about 10%, and more preferably less than about 6%, it is expected that the stent 30 will not be permanently deformed, but rather may return to its preconfigured heat-set shape, as explained in further detail below.

In a next step, the stent 30 may be exposed to a reduced temperature environment to temporarily hold the stent in the expanded state shown in FIG. 2. By way of example, the stent 30 may be cooled using liquid nitrogen, cooled alcohol, or the Quik-Freeze® coolant by Miller-Stephenson company of Sylmar, Calif. The stent may be exposed to the reduced temperature environment using any suitable technique, including but not limited to spraying a cooling substance onto the stent, immersing the stent into a bath of a cooling substance, cooling the room temperature, and so forth. Exemplary techniques for cooling the stent 30, which are not intended to be limiting, are described further below.

In one example, a cooling substance may be provided in the form of chilled fluid capable of placing a shape-memory stent into a martensitic state. The cooling substance therefore may be capable of imparting a temperature change to the stent 30. In some embodiments, the fluid may comprise a gas, such as air, and an inert gas, such as nitrogen. In other embodiments, the fluid may comprise a liquid. For example, liquid nitrogen may be used and may be supplied at temperatures of −196 degrees Celsius or less.

The stent 30 may be cooled using a commercially available loading chamber with a commercially available liquid nitrogen source attached thereto. One suitable loading chamber, which is coupled to a liquid nitrogen source, is described in further detail in U.S. Pat. Pub. No. 2006/0196073 (“the '073 publication”), which is commonly assigned with the present application, and is hereby incorporated by reference in its entirety. As explained in the '073 publication, a stent may be loaded into the chamber and the temperature of the chamber and device is lowered by a liquid nitrogen source that may be supplied to the chamber.

In another example, a suitable cooling medium may be provided in the form of a spray sold under the name Envi-Ro-Tech™ Freezer, manufactured by Techspray, L.P., of Amarillo, Tex. This formulation may be non-cytotoxic when sprayed onto stents, and evaporates quickly and leaves no trace chemicals on the stent. The formulation may cause the stent to obtain a temperature below a transition temperature, such as the martensitic start temperature of nitinol.

In addition to using a cooling substance, or as an alternative, the ambient temperature in the room may be lowered to a temperature to facilitate retention of the stent 30 below a transition temperature, such as the martensitic start temperature of nitinol. Moreover, one or more of the above-mentioned cooling techniques may be employed while the stent 30 is still coupled to the inflated balloon 54, as shown in FIG. 2, or may be applied after the stent is uncoupled from the balloon 54, for example, by deflating the balloon 54.

After the stent has been expanded to the larger inner diameter d₂, and then cooled to help retain the expanded larger diameter, the stent 30 may be temporarily retained in the second expanded state at the inner diameter d₂, which is larger than the outer diameter d_{c} of the coating 40 and the preconfigured, first expanded state having the inner diameter d₁. Subsequently, the stent 30 may be securely coupled to the coating 40, as explained in further detail in FIGS. 4-5 below.

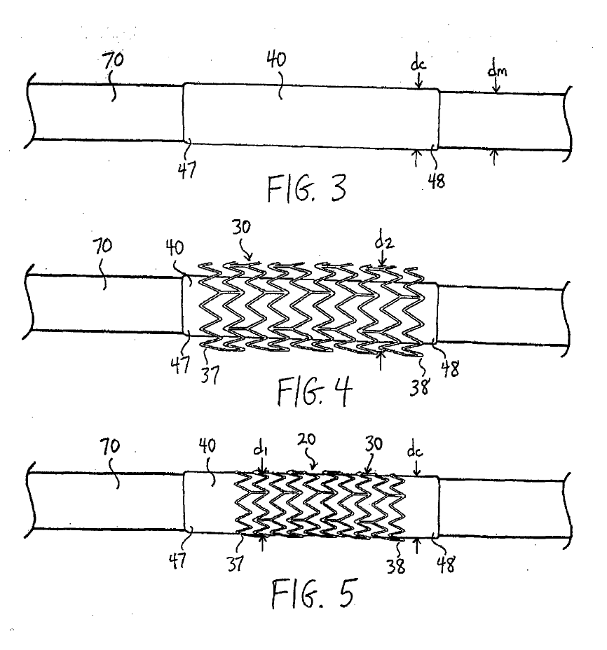

Referring now to FIG. 3, the coating 40 is shown prior to being coupled to the expanded stent 30 of FIG. 2. In one embodiment, an inert mandrel 70 having an outer diameter dm, such as a glass mandrel, may be immersed or sprayed with a composition to form the coating 40 having a desired configuration. In one example, the mandrel 70 may be cleaned with isopropyl alcohol. The composition of the coating 40 may be prepared by dissolving a polymer in a solvent including alcohols, aromatic hydrocarbons, dimethyl acetamide (DMAC), and the like. The composition may be varied to obtain the desired viscosity of the coating 40. If Thoralon® is the selected polymer, as noted above, and DMAC is the selected solvent, the polymer may comprise about 5% to about 40% by weight of the total weight of the composition.

In one example, the mandrel 70 may be immersed in the composition at a predetermined speed through a die, and the solvent then can be removed or allowed to evaporate to form a film layer of the coating 40 on the mandrel 70, as shown in FIG. 3. Further, evaporation of the solvent can be induced by application of heat treatment for about 5 minutes to about 24 hours in an oven having a temperature of about 25 to about 80 degrees Celsius. Alternatively, vacuum conditions may be employed. The finished coating 40 comprises an outer diameter d_{c} and has first and second ends 47 and 48, as shown in FIG. 3.

Referring now to FIG. 4, in a next step, the stent 30 may be placed over the mandrel 70 and aligned in a generally coaxial manner with respect to the coating 40. As explained with respect to FIG. 2 above, the stent 30 may be expanded to have an inner diameter d₂, which is greater than the outer diameter d_{c} of the coating 40, as depicted in FIG. 4. Since the stent 30 may be temporarily retained in the larger diameter state, e.g., using the cooling techniques described herein, it may be easier to co-axially align the stent 30 over the coating 40.

Referring now to FIG. 5, the stent 30 may be allowed to return to the preconfigured first expanded state. The stent and the coating may be placed in an increased temperature environment, relative to the reduced temperature environment noted above. The increased temperature environment, which may comprise room temperature conditions or a heat source, may cause the stent 30 to return to its original heat-set shape. In the first expanded state, the stent 30 has an inner diameter d₁, which preferably is less than or equal to the outer diameter d_{c}, of the coating 40, as depicted in FIG. 5. The stent 30 therefore is allowed to securely form around the coating 40, thereby forming the coated stent 20 of FIGS. 1 and 5. Once formed, the coated stent 20 may be removed from the mandrel 70. The first and second ends 47 and 48 of the coating 40 may be trimmed or modified to comport to the shapes of the corresponding ends 37 and 38 of the stent 30, as depicted in FIG. 1.

Advantageously, since the stent 30 has a preconfigured inner diameter d₁ that is less than or equal to the outer diameter d_{c} of the coating 40, gaps between the stent 30 and the coating 40 may be reduced or eliminated. If the coating 40 comprises Thoralon®, which has a relatively soft and sticky nature, the inner surfaces 35 of the stent 40 may become at least partially embedded into the outer surface 42 of the coating 40. Therefore, the need for a separate adhesive to couple the stent 30 to the coating 40 may be avoided.

Moreover, since potential gaps between the stent 30 and the coating 40 are substantially reduced or eliminated, there may be no need to place an additional coating over the stent 30. By reducing the number of coatings or layers coupled to the stent 30, the stent 30 may comprise a less bulky profile and the force necessary to deploy the stent 30 may be reduced.

The coated stent 20 may be delivered into a vessel, duct, or other anatomical site using a suitable deployment system or introducer. An introducer, such as that described in PCT application WO98/53761, entitled “A Prosthesis and a Method and Means of Deploying a Prosthesis,” which is incorporated herein by reference in its entirety, may be used to deploy the stent-grafts. PCT application WO98/53761 describes a deployment system for an endoluminal prosthesis whereby the prosthesis is radially compressed onto a delivery catheter and is covered by an outer sheath. To deploy the system, the operator slides or retracts the outer sheath over the delivery catheter, thereby exposing the prosthesis. The prosthesis expands outwardly upon removal of the sheath. The operator can directly manipulate the sheath and the delivery catheter, which provides the operator with a relatively high degree of control during the procedure. Further, such delivery devices may be compact and may have a relatively uniform, low-diameter radial profile, allowing for atraumatic access and delivery.

As noted above, in other examples, the stent 30 may comprise other shapes. Further, multiple stents 30 may be provided and individually coupled to the coating 40. For example, several individual nitinol Z-stents may be mechanically expanded, cooled, and then disposed over the coating 40 in the manner described above, thereby securing multiple separate rings to the coating 40. Similarly, one or more stents may be circumferentially wound in a continuous fashion to form a coil or helical wire structure, and then attached to the coating 40 using the techniques described herein. In each instance, since an inner diameter of the stent is less than or equal to an outer diameter of the coating, gaps between the stent and coating may be reduced and the need for an additional coating may be eliminated.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

I claim:

1. A coated stent comprising:
a coating comprising an outer surface having an outer diameter; and
a stent having a compressed state and a first expanded state,
where the stent is disposed over the coating, and
where an inner diameter of the stent in the first expanded state is less than or equal to the outer diameter of the coating to cause an inner surface of the stent to engage the outer surface of the coating, and where the inner surface of the stent is at least partially embedded in the coating.

2. The coated stent of claim 1, where an outer surface of the stent is substantially free of any coating.

3. The coated stent of claim 1, where the coating comprises a porous polymeric sheet that comprises a polyetherurethane urea.

4. The coated stent of claim 1, where the stent comprises a shape-memory material that is preconfigured to expand to the first expanded state.

5. The coated stent of claim 4, where the stent comprises a nickel-titanium alloy.

6. The coated stent of claim 1, where the stent comprises a second expanded state in which the inner diameter of the stent is greater than the outer diameter of the coating, and where the stent is adapted to be placed over the coating in the second expanded state prior to being coupled to the coating.

7. The coated stent of claim 6, where the stent is adapted to be expanded to the second expanded state using a balloon catheter.

8. The coated stent of claim 6 further comprising a cooling substance adapted to temporarily retain the stent in the second expanded state.

9. A coated stent comprising:
a coating comprising an outer surface having an outer diameter; and
a stent comprising a shape-memory material, where the stent further comprises a compressed state and a first expanded state having an inner diameter less than or equal to the outer diameter of the coating, where the stent is preconfigured to expand to the first expanded state,
where the stent further comprises a second expanded state in which the inner diameter of the stent is greater than the outer diameter of the coating, and
where the stent is adapted to be placed over the coating in the second expanded state and adapted to return to the first expanded state to cause an inner surface of the stent to engage the outer surface of the coating.

10. The coated stent of claim 9, where an outer surface of the stent is substantially free of any coating.

11. The coated stent of claim 9, where the coating comprises a porous polymeric sheet that comprises a polyetherurethane urea.

12. The coated stent of claim 9, where the stent comprises a nickel-titanium alloy.

13. The coated stent of claim 9, where the stent is adapted to be expanded to the second expanded state using a balloon catheter.

14. A method suitable for coupling a stent to a coating, the method comprising:
providing a coating comprising an outer surface having an outer diameter;
providing a stent having a compressed state and a first expanded state, wherein an inner diameter of the stent in the first expanded state is less than or equal to the outer diameter of the coating;
increasing the diameter of the stent to a second expanded state in which the inner diameter of the stent is greater than the outer diameter of the coating;
placing the stent over the coating in the second expanded state; and
allowing the stent to return to the first expanded state to cause an inner surface of the stent to engage the outer surface of the coating.

15. The method of claim 14, where increasing the diameter of the stent comprises inflating the stent using a balloon catheter.

16. The method of claim 14, where the stent comprises a shape-memory material that is preconfigured to expand to the first expanded state, the method further comprising exposing the stent to a reduced temperature environment in the second expanded state to facilitate placement of the stent over the coating in the second expanded state.

17. The method of claim 16, where allowing the stent to return to the first expanded state comprises placing the stent and the coating in an increased temperature environment, relative to the reduced temperature environment, to cause the shape-memory material to return to the preconfigured first expanded state.

18. The method of claim 16, where exposing the stent to a reduced temperature environment comprises exposing the stent to liquid nitrogen.

19. The method of claim 16, where exposing the stent to a reduced temperature environment comprises spraying a cooling substance onto the stent.

20. The method of claim 14 further comprising:
providing a mandrel;
forming the coating over an exterior surface of the mandrel;
placing the stent over the coating on the mandrel when the stent is in the second expanded state; and
removing the coated stent from the mandrel after the stent returns to the first expanded state.
